(19) United States
(12) Patent Application Publication
(10) Pub. No.: US 2025/0152478 A1
(43) Pub. Date: May 15, 2025

(54) WET-SETTING ADHESIVES FOR ORAL ENVIRONMENT AND METHODS OF USE THEREOF

(71) Applicants: Purdue Research Foundation, West Lafayette, IN (US); Colgate-Palmolive Company, New York, NY (US)

(72) Inventors: Jonathan James Wilker, West Lafayette, IN (US); Future Mayurkumar Jani, Ahmedabad (IN); Emma Claire Jeffries, Greenwood, IN (US); Julie Liu, West Lafayette, IN (US); Aishwarya Vijayan Menon, Lafayette, IN (US); Gudrun Schmidt, West Lafayette, IN (US); Myah Diana Webb, Camden, TN (US); Joseph Steele, Plainfield, NJ (US); Lin Fei, Kendall Park, NJ (US)

(21) Appl. No.: 18/946,278

(22) Filed: Nov. 13, 2024

Related U.S. Application Data
(60) Provisional application No. 63/548,243, filed on Nov. 13, 2023.

(51) Int. Cl. A61K 6/30; A61K 6/898; C09J 105/00; C09J 189/00

(52) U.S. Cl. CPC A61K 6/30; A61K 6/898; C09J 105/00; C09J 189/005; C09J 2400/10; C09J 2400/20; C09J 2405/00; C09J 2489/00

(57) ABSTRACT

Methods of using wet-setting adhesives in oral environments, methods of using adhesives for bonding a dental material to a tooth, a gum, or a soft tissue in a mouth, a method of sealing a wound or closing an incision in a gum or a soft tissue, and a method of forming an adhesive coating on a tooth.

### CROSS-REFERENCE TO RELATED APPLICATIONS

This application claims priority to U.S. provisional patent application No. 63/548,243, which was filed Nov. 13, 2023, and which is hereby incorporated by reference in its entirety.

### TECHNICAL FIELD

The present disclosure relates to methods of wet-setting adhesives in an oral environment and, in particular, methods of using wet-setting adhesives that provide a strong bonding in saliva and saline solution.

### BACKGROUND

This section introduces aspects that may help facilitate a better understanding of the disclosure. Accordingly, these statements are to be read in this light and are not to be understood as admissions about what is or is not prior art.

Bonding to surfaces underwater is difficult, and bonding to biofilm-covered surfaces underwater can be even more difficult or not possible. Nontoxic and plant-based adhesives that are effective on wet surfaces are appealing and needed for applications in the oral and dental environment. Adhesives that stick in wet oral environments, at body temperatures, and in the presence of saliva, biofilms and other fouled surfaces are rare or nonexistent. Various kinds of metal materials, organic polymer materials, and ceramic materials are conventionally used for the restoration of teeth. When these restorative materials are mounted in the mouth, adhesion is necessary between the teeth and the metal, organic polymer, or ceramic material, as is adhesion of the restorative materials to each other, for example, metal to metal, ceramics, or organic polymer. In particular, since they are used in the mouth in the dentistry field, it would be beneficial for adhesion to be satisfactory under wet conditions. Despite this, conventional dental adhesives require rigorous cleaning and drying of teeth prior to use.

Thus, it is an object of the present disclosure to provide methods of using wet-setting adhesives which provide strong bonding in an oral environment. This and other objects and advantages, as well as inventive features, will be apparent from the detailed description.

### SUMMARY

Provided is a method for bonding a dental material to a tooth surface, which method comprises:
applying an adhesive comprising (i) a zein, (ii) a tannic acid and (iii) optionally a filler to either (a) the tooth surface, (b) the dental material, or (c) both (a) and (b);
applying the dental material onto the tooth surface; and
allowing the adhesive to cure to fix the dental material to the tooth surface, whereupon the dental material is bonded to the tooth surface.

The dental material can comprise a dental restorative or an orthodontic appliance. In some embodiments, the dental restorative is a composite, a filling, a sealant, an inlay, an onlay, a crown, or a bridge. In some embodiments, the orthodontic appliance is a bracket, a crown, a buccal tube, a band, a cleat, a button, a lingual retainer, or a lingual bar. In some embodiments, the filler is an inorganic component. In some embodiments, the filler is a polymer.

The inorganic component used in the filler of the adhesive can be selected from a group consisting of natural clay, calcium carbonate (CaCO₃), zinc Oxide (ZnO), synthetic clay, and any combination of the foregoing. In some embodiments, the inorganic component is selected from Montmorillonite (MMT-K10); Montmorillonite, dimethyl dialkyl amine (MMT-DDA or MMT-amine); Montmorillonite, trimethyl stearyl ammonium (MMT-TSA or MMT-am); Laponite® RD (LRD), Laponite® XLS; ZnO, and CaCO₃.

The polymer used in the adhesive can be selected from protein, polysaccharide, and a combination thereof. In some embodiments, the polymer is selected from casein, albumin, soy, gelatin, mucin, hydroxypropyl methyl cellulose, methyl cellulose, α-cellulose, Avicel pH-10, and any combination thereof.

Provided is a method for bonding a dental material to a gum or a soft tissue in a mouth, which method comprises:
applying an adhesive comprising (i) a zein, (ii) a tannic acid and (iii) optionally a filler either (a) to the dental material, (b) to the gum or the soft tissue in the mouth, or (c) both (a) and (b);
applying the dental material onto the gum or the soft tissue in the mouth; and
allowing the adhesive to cure to fix the dental material to the gum or the soft tissue in the mouth, whereupon the dental material is bonded to the gum or the soft tissue in the mouth.

In some embodiments, the dental material bonded to the gum or soft tissue is a composite, a filling, or a sealant. The filler can be an inorganic component or a polymer, as described above.

Still further provided is a method of sealing a wound or closing an incision in a gum or a soft tissue of a mouth, which method comprises: applying an adhesive comprising (i) a zein, (ii) a tannic acid, and (iii) an inorganic component to the wound or the incision in the gum or the soft tissue of the mouth, whereupon the wound is sealed or the incision is closed.

Still further provided is a method of sealing a wound or closing an incision in a gum or a soft tissue of a mouth, which method comprises: applying an adhesive comprising (i) a zein, (ii) a tannic acid, and (iii) a polymer to the wound or the incision in the gum or the soft tissue of the mouth, whereupon the wound is sealed or the incision is closed.

Provided is a method of forming a coating on a tooth surface, which method comprises:
applying an adhesive comprising (i) a zein, (ii) a tannic acid, and (iii) optionally a filler to the tooth surface; and
allowing the adhesive to cure, whereupon the adhesive forms a coating on the tooth surface.

In some embodiments, zein is at or about 25-45 weight percent, and the tannic acid present in at or about 1-25 weight percent of the cured coating. The filler can be an inorganic component or a polymer, as described above.

### BRIEF DESCRIPTION OF THE DRAWINGS

The present disclosure will be more readily understood from the detailed description of embodiments presented below, considered in conjunction with the attached drawings of which:

FIG. 1 shows graphical data of the adhesive strength (MPa) under saline solution, obtained from lap shear testing, of adhesive containing (i) zein (Z) and tannic acid (TA) (control), (ii) Z 24%, TA 33%, and Laponite® RD (LRD) 43% (diagonal striped), and (iii) Z 18%, TA 25%, and Laponite® XLS (XLS) 57% (horizontal striped). All adhesives were kept in saline solution for 24 hours at 37° C. prior to lap shear testing. At least five data points were used for averaging. Weight percentage (wt %) refers to the dry solid content of materials without solvents. The adhesive compositions were each tested on four substrates, e.g., limestone, stainless steel, collagen, and hydroxyapatite.

FIG. 2 shows graphical data of the bond strengths, obtained from lap shear testing, in saline solution for an adhesive containing zein, tannic acid, and calcium carbonate (Z 24%-TA 33%-CaCO₃ 43%). The adhesive was tested on four substrates, e.g., limestone, stainless steel, collagen, and hydroxyapatite, each against a control (Z 42%-TA 58%). After lap-shear testing, limestone, stainless steel, and hydroxyapatite substrates showed both adhesive and cohesive failure modes with a little more cohesive failure. Collagen substrates showed adhesive failure most of the time.

FIG. 3 shows graphical data of the adhesive strength under-saline solution, obtained from lap shear testing, of adhesive compositions containing zein, tannic acid, and polymer additives. Adhesives tested were (i) Z 41%-TA 57%-HPMC 2% (diagonal stripes), (ii) Z 30%-TA 42%-Albumin 28% (vertical stripes), and (iii) Z 42%-TA 58%-control (plain). The adhesive compositions were each tested on four substrates, e.g., limestone, stainless steel, collagen, and hydroxyapatite. At least 5 data points were used for averaging in each instance. The adhesives were kept in saline solution for 24 hours at 37° C. prior to lap shear testing. Limestone, stainless steel, and hydroxyapatite substrates showed both adhesive and cohesive failure modes with a little more cohesive failure. Collagen substrates showed adhesive failure most of the time.

FIG. 4 shows a graph of adhesion strengths in artificial saliva solutions obtained from lap shear testing of adhesives containing zein, tannic acid, and inorganic component: (i) Z 24%-TA 33%-LRD 43% and (ii) Z 24%-TA 33%-CaCO₃ 43%. Wt % refers to the dry solid materials without solvents. The application of each adhesive was done in artificial saliva. At least 5 data points were used for averaging in each instance. The adhesives were tested on collagen and hydroxyapatite substrates.

FIG. 5 shows images of a drop of highly viscous zein-tannic acid adhesive (Z 42%-TA 58%) sandwiched between glass slides and then exposed to artificial saliva for 100 minutes at room temperature. In principle, the glue slowly cures when exposed to the solution, which results in the ring around the glue becoming thicker over time. As the artificial saliva evaporated, deionized water was added between glass slides to avoid drying and to maintain the same solution composition. The size of the image shown is 22 mm, the size of the microscopy cover slide.

FIG. 6 shows a graph of the adhesion strengths of Z 42%-TA 58% adhesive cured for 1 hour at temperatures between 30° C. to 45° C. in artificial saliva using hydroxyapatite substrates.

FIG. 7 shows the adhesion strength of the Z-TA adhesive in artificial saliva at 37° C. on bronze substrates versus tannic acid content in wt % of solid. The curing time was 1 hour. The y-axis shows the averaged adhesion strength calculated, and the x-axis shows the tannic acid content in wt % solid in the Z-TA adhesives. Corresponding Z-TA compositions are listed in Tables 1-2. Average mean values and standard deviations are shown for at least five samples.

FIG. 8A shows a graph of the adhesion strength of control Z-TA (Z 42%-TA 58%) and Z-TA-zinc oxide (ZnO) (Z 26%-TA 37%-ZnO) 37%) adhesive versus adhesive curing time in artificial saliva at 37° C. The substrates were hydroxyapatite. Sample adhesive compositions are listed in Table 2.

FIG. 8B shows a graph of the adhesion strength of Z-TA-ZnO Vs. ZnO content in wt % solid. Curing time is one hour in artificial saliva at 37° C. The substrates were hydroxyapatite. Sample adhesive compositions are also listed in Table 2.

FIG. 9 shows the adhesion strength of the adhesive Z 24%-TA 33%-LRD 43% and selected data points for control, Z-TA (Z 42%-TA 58%), as a function of different curing times. Hydroxyapatite substrates were used in artificial saliva.

FIG. 10A shows the adhesion strength of Z 69%-TA 31% on bronze substrates versus the time these substrates spent in artificial saliva before adhesive was applied. The curing time of glued substrates was 1 hour at 37° C. Average mean values and standard deviations are shown for at least five samples.

FIG. 10B shows the adhesion strength of Z 42%-TA 58% on bronze substrates versus time these substrates spent in artificial saliva before adhesive was applied. Curing time of glued substrates was 1 hour at 37° C. Average mean values and standard deviations are shown for at least five samples.

FIG. 11A shows the images of the commercial PreviDent® varnish coating on substrates before and after water flosser test at pressure 1.

FIG. 11B shows the images of the Colgate® Optic White® Overnight Teeth Whitening Pen coating on substrates before and after the water flosser test at pressure 1.

FIG. 11C shows the images of the POLYGRIP® denture adhesive coating on substrates before and after the water flosser test at pressure 1.

FIG. 12A shows the images of the coating formed by adhesive Z35T14 with 1 wt % ZnO on substrates before and after the water flosser test at pressure 1.

FIG. 12B shows the images of the coating formed by adhesive Z35T14 with 5 wt % ZnO on substrates before and after the water flosser test at pressure 1.

FIG. 12C shows the images of the coating formed by adhesive Z35T21 with 1 wt % ZnO on substrates before and after the water flosser test at pressure 1.

### DETAILED DESCRIPTION

For the purposes of promoting an understanding of the principles of the present disclosure, reference will now be made to the embodiments illustrated in the drawings, and specific language will be used to describe the same. No limitation of scope is intended by the description of these embodiments. On the contrary, this disclosure is intended to cover alternatives, modifications, and equivalents as may be included within the spirit and scope of this application as defined by the appended claims.

The terms “adhesive” and “adhesive composition” are used interchangeably.

The term “dental material” refers to a material that can be bonded to a dental surface (e.g., tooth), a soft tissue in the mouth (gum), or a mucosa and includes, for example, dental restoratives and orthodontic appliances.

The term “dental restorative” refers to a material used to repair or replace a tooth, or portion thereof, or to change the appearance of the tooth. The dental restorative material can be any suitable material as is known in the art. Examples include, but are not limited to, metal material, polymeric material, and ceramic material.

The term “curing” refers to loss of solvent, polymerization, and/or crosslinking reactions.

As difficult as underwater bonding may be, in general, attaching wet surfaces that are fouled with organic compounds is even more difficult. The adhesive compositions comprising zein and tannic acid are strong underwater adhesives. However, in the oral environment, saliva plays an essential role in controlling wetness in the mouth, which contains high concentrations of many organic compounds (including mucin and albumin, for example), various enzymes, and antibacterial compounds. The oral environment is influenced by local factors, including pH of saliva, what a person cats and/or drinks, oral microflora, dental plaque, and tartar. It is difficult for any adhesive to function in such a wet and fouled environment. The materials used for oral application are desirably biocompatible, durable, nonreactive in acid and alkaline conditions, and compatible with other dental materials that are used, such as dental material, dental restoratives, and orthodontics appliances.

The wet-setting adhesives hereof can provide a strong bond in the oral environment and can stick well even in the presence of biofilms. Thus, the adhesives can be used as a dental adhesive, a sealant, a cement, a primer and/or for bonding or cementing a material to the surface of the dental tissue. For example, the adhesives can be used for bonding a dental material to a tooth or a gum or a soft tissue in a mouth of a subject. A “subject” can be a mammal. For example, and without limitation, the subject can be a human, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale.

The adhesives can be used for bonding and fixing a tooth (e.g., of a subject) and a dental material, e.g., inlay, onlay, abutment tooth, bridge, post, splint, orthodontic bracket, crown, etc., or bonding dental restoratives to each other, e.g., abutment tooth and crown.

Provided is a method for bonding a dental material to a tooth surface, which method comprises:
applying, to either (a) the tooth surface, (b) the dental material, or (c) both (a) and (b), an adhesive comprising a zein, a tannic acid and, optionally, a filler;
applying the dental material onto the tooth surface; and
allowing the adhesive to cure and fix the dental material to the tooth surface, whereupon the dental material is bonded to the tooth surface.

In certain embodiments, curing the adhesive (e.g., allowing the adhesive to cure) can facilitate crosslinking within at least the macromolecule of the adhesive, thus promoting strong adhesive properties. In some embodiments, the filler can be an inorganic component. In some embodiments, the filler can be a polymer.

Zein is an alcohol-soluble prolamine protein present in corn or maize. Pure zein can be yellow, non-toxic, and water-insoluble powder. Zein is a naturally degradable material with a strong hydrophobicity. The amino acid sequence of naturally occurring (i.e., native) zein is publicly available. Tannic acid is a naturally occurring polyphenol that comprises reactive terminal phenolic hydroxyl groups that interact with the zein protein functional groups and together balance cohesion and adhesion in a way that the adhesive sticks well on a wet surface. The amount of tannic acid used in the adhesive composition can be higher than zein. Zein can act as the matrix or binder that crosslinks and holds the tannic acid molecules together. The compositions high in tannic acid concentrations stick well on the wet surface and cannot be easily removed. The inorganic component can improve the adhesive strength by forming bonds between zein and tannic acid. The inorganic component can be added to the zein-tannic acid adhesive to increase the adhesion, tailor viscosity, simplify handling procedures, and control curing times. Depending on the composition, viscosities of the adhesives can range between low and high viscous flowing solutions, gel-like glues that hold their weight, viscoelastic putty materials, and cement-like adhesives.

In some embodiments, the adhesive used in the methods hereof comprises an adhesive described in U.S. patent application Ser. No. 18/720,469, which is incorporated by reference in its entirety herein. In some embodiments, the adhesive used in the methods hereof comprises an adhesive described in International Patent Application Publication No. WO2024/040270, which is incorporated by reference in its entirety herein. In certain embodiments, the adhesives of either of the aforementioned patent application or publication can be modified as described herein.

In some embodiments, the zein-tannic acid (Z-TA) adhesive comprises zein in an amount of about 30 wt % dry solid to about 100 wt % dry solid. In some embodiments, the Z-TA adhesive comprises zein in the amount of about 30 wt % of dry solid. In some embodiments, the Z-TA adhesive comprises zein in the amount of about 35 wt % dry solid. In some embodiments, the Z-TA adhesive comprises zein in the amount of about 39 wt % dry solid. In some embodiments, the Z-TA adhesive comprises zein in the amount of about 42 wt % dry solid. In some embodiments, the Z-TA adhesive comprises zein in the amount of about 47 wt % dry solid. In some embodiments, the Z-TA adhesive comprises zein in the amount of about 52 wt % dry solid. In some embodiments, the Z-TA adhesive comprises zein in the amount of about 60 wt % dry solid. In some embodiments, the Z-TA adhesive comprises zein in the amount of about 69 wt % dry solid. In some embodiments, the Z-TA adhesive comprises zein in the amount of about 81 wt % dry solid. In some embodiments, the Z-TA adhesive comprises zein in the amount of about 88 wt % dry solid. In some embodiments, the Z-TA adhesive comprises zein in the amount of about 96 wt % dry solid. All wt % set forth in this paragraph reference the wt % of the uncured adhesive and all ranges are inclusive of the stated end points and all 1% increments therein.

In some embodiments, the Z-TA adhesive comprises tannic acid in an amount of about 0 wt % to about 70 wt % dry solid. In some embodiments, the Z-TA adhesive comprises tannic acid in the amount of about 4 wt % dry solid. In some embodiments, the Z-TA adhesive comprises tannic acid in the amount of about 12 wt % dry solid. In some embodiments, the Z-TA adhesive comprises tannic acid in the amount of about 19 wt % solid. In some embodiments, the Z-TA adhesive comprises tannic acid in the amount of about 31 wt % dry solid. In some embodiments, the Z-TA adhesive comprises tannic acid in the amount of about 40 wt % dry solid. In some embodiments, the Z-TA adhesive comprises tannic acid in the amount of about 48 wt % dry solid. In some embodiments, the Z-TA adhesive comprises tannic acid in the amount of about 53 wt % dry solid. In some embodiments, the Z-TA adhesive comprises tannic acid in the amount of about 58 wt % dry solid. In some embodiments, the Z-TA adhesive comprises tannic acid in the amount of about 61 wt % dry solid. In some embodiments, the Z-TA adhesive comprises tannic acid in the amount of about 65 wt % dry solid. In some embodiments, the Z-TA adhesive comprises tannic acid in the amount of about 70 wt % dry solid. All wt % set forth in this paragraph reference the wt % of the uncured adhesive.

Provided is a method for bonding a dental material to a tooth surface, which method comprises:
applying an adhesive comprising a zein, a tannic acid, and an inorganic component to either (a) the tooth surface, (b) the dental material, or (c) both (a) and (b);
applying the dental material onto the tooth surface; and
allowing the adhesive to cure and, thus, fix the dental material to the tooth surface, whereupon the dental material is bonded to the tooth surface.

The tissues of the tooth surface can include enamel, dentin, cementum, and pulp. While the pulp is in the center of the tooth and contains nerves, blood vessels, and connective tissue, the enamel is on the exposed surface of the tooth. Dentin is the layer below the enamel. Cementum is on the surface of the tooth below the gumline. The surface of the tooth can be wet due to the presence of saliva in the mouth. No external wetness need be applied. In some embodiments, the adhesive can be applied to the dental material, and the dental material having the adhesive thereon applied to the tooth surface. In some embodiments, the adhesive can be applied to the tooth surface on which the dental material can be placed. In some embodiments, the adhesive can be applied to both the tooth surface and the dental material.

The dental material can be a dental restorative or an orthodontic appliance. Dental restoratives include, but are not limited to, composites, fillings, sealants, inlays, onlays, abutment teeth, posts, splints, crowns, and bridges. In some embodiments, the restorative is a composite resin, glass ionomer, compomer, giomer, Orcomer®, or any other material composed of polymers and/or calcium silicate, glass, or ceramic.

Orthodontic appliances include, but are not limited to, brackets, crowns, buccal tubes, bands, cleats, buttons, lingual retainers, and lingual bars.

The adhesive used can be a bio-based adhesive that strongly bonds (e.g., by curing) the tooth surface and the dental material in an oral environment. In some embodiments, the adhesive comprises (i) a zein, (ii) a tannic acid, and (iii) an inorganic component, wherein the adhesive comprises about 40-80 wt % of the inorganic component (wt % of uncured adhesive).

In some embodiments, the amount of the inorganic component present in the adhesive is about 40-70 wt % (of uncured adhesive). In some embodiments, the amount of the inorganic component is about 40-65 wt % (of uncured adhesive). In some embodiments, the amount of the inorganic component is about 40-60 wt % (of uncured adhesive).

Suitable inorganic components, as are known in the art, can be used. Examples of suitable inorganic components include, but are not limited to, natural clay, calcium carbonate (CaCO₃), zinc oxide (ZnO), synthetic clay, and any combination thereof. Examples of natural clays include, but are not limited to, Montmorillonite (MMT-K10); Montmorillonite, dimethyl dialkyl amine (MMT-DDA or MMT-amine); and Montmorillonite, trimethyl stearyl ammonium (MMT-TSA or MMT-am). Laponite® is a non-limiting example of a synthetic clay. Laponite® is a well-known synthetic silicate clay used in many applications in the pharmaceutical and cosmetic sectors. Laponite® RD (LRD) is composed of pure clay silicate nanoplates, and Laponite® XLS (XLS) is a low-heavy metal polyphosphate-treated laponite clay. LRD provides charged silicate nanoplates, such as the surfaces of the round nanoplatelets being negatively charged and the sides being positively charged that readily interact with the polar functional groups within the zein protein and the tannic acid. These hydrophilic characteristics of LRD are responsible for polymer absorption and water swelling properties. Thus, Laponite® can enhance the cohesive properties of the material, while tannic acid can be responsible for the wet or underwater adhesion to surfaces. Calcium carbonate is a non-toxic natural material that can increase the bond strength of zein-tannic acid adhesive for dental materials. In some embodiments, the inorganic component is CaCO₃, ZnO, or Laponite®. Zinc oxide can be used as an inorganic component and for reducing curing times.

Provided is a method for bonding a dental material to a tooth surface, which method comprises:
applying an adhesive comprising a zein, a tannic acid, and a polymer to either (a) the dental material, (b) the tooth surface, or (c) both (a) and (b);
applying the dental material onto the tooth surface; and
allowing the adhesive to cure and fix the dental material to the tooth surface, whereupon the dental material is bonded to (i.e., affixed to) the tooth surface.

In some embodiments, the adhesive can be applied to the dental material, and the dental material having the adhesive thereon applied to the tooth surface. In some embodiments, the adhesive can be applied to the tooth surface on which the dental material can be placed. In some embodiments, the adhesive can be applied to both the tooth surface and to the dental material. The dental material can be a dental restorative or an orthodontic appliance. Dental restoratives include, but are not limited to, composites, fillings, sealants, inlays, onlays, abutment teeth, posts, splints, crowns, and bridges. In some embodiments, the restorative is a composite resin, glass ionomer, compomer, giomer, Orcomer®, or any other material composed of polymers and/or calcium silicate, glass, or ceramic. Orthodontic appliances include, but are not limited to, brackets, crowns, buccal tubes, bands, cleats, buttons, lingual retainers, and lingual bars.

In some embodiments, the adhesive comprises (i) a zein, (ii) a tannic acid, and (iii) a polymer, wherein the adhesive comprises about 2-80 wt % of polymer (of the uncured adhesive).

In some embodiments, the amount of polymer present in the adhesive is about 2-70 wt % (of the uncured adhesive). In some embodiments, the amount of the polymer is about 2-60 wt % (of the uncured adhesive). In some embodiments, the amount of the polymer is about 2-50 wt %. In some embodiments, the amount of the polymer is about 2-40 wt % (of the uncured adhesive). In some embodiments, the amount of the polymer is about 2-30 wt % (of the uncured adhesive).

In some embodiments, the polymer used is a natural polymer. Natural polymer used in the adhesive can be a protein, a polysaccharide, or a combination thereof. Examples of polysaccharides include, but are not limited to, cellulose derivatives such as hydroxypropyl methyl cellulose (HPM), methylcellulose (M Cell), α-cellulose (α Cell), Avicel PH-101 and any combination thereof. Examples of proteins include, but are not limited to, casein, albumin, soy, gelatin, mucin, and any combination thereof. The soy protein can be selected from soybean flour, soy protein isolate, soy protein acid hydrolysate, and gelatin. In various embodiments, albumin is a preferred protein. Albumin is a cross-linkable protein. In various embodiments, cellulose derivatives are a preferred polysaccharide.

In some embodiments, the Z-TA-filler adhesive comprises zein in an amount of about 15 wt % of dry solid to about 40 wt % of dry solid, such as about 15 wt % of dry solid to 40 wt % of dry solid or 15 wt % of dry solid to about 40 wt % of dry solid or 15 wt % of dry solid to 40 wt % of dry solid.

In some embodiments, the Z-TA-filler adhesive comprises tannic acid in an amount of about 20 wt % of dry solid to about 55 wt % of dry solid, such as about 20 wt % of dry solid to 55 wt % of dry solid or 20 wt % of dry solid to about 55 wt % of dry solid or 20 wt % of dry solid to 55 wt % of dry solid. All wt % set forth in this paragraph reference the wt % of the uncured adhesive and all ranges are inclusive of the stated end points and all 1% increments therein.

In some embodiments, the Z-TA-filler adhesive comprises the filler in an amount of about 2 wt % of dry solid to about 80 wt % of dry solid, such as about 2 wt % of dry solid to 80 wt % of dry solid or 2 wt % of dry solid to about 80 wt % of dry solid or 2 wt % of dry solid to 80 wt % of dry solid.

Adhesives hereof can also form a strong bond between dental materials and gum or soft tissues all around or in the mouth. Provided is a method for bonding a dental material to a gum or a soft tissue in a mouth of a subject, which method comprises:
applying, either (a) to the dental material, (b) to the gum or the soft tissue in the mouth, or (c) both (a) and (b), an adhesive comprising a zein, a tannic acid, and, optionally a filler;
applying the dental material onto the gum or the soft tissue in the mouth; and
allowing the adhesive to cure and fix the dental material to the gum or the soft tissue in the mouth, whereupon the dental material is bonded (i.e. or affixed) to the gum or the soft tissue in the mouth.

In some embodiments, the filler can be an inorganic component. In some embodiments, the filler can be a polymer.

Provided is a method for bonding a dental material to a gum or a soft tissue in a mouth, which method comprises:
applying an adhesive comprising a zein, a tannic acid, and an inorganic component either (a) to the dental material, (b) to the gum or the soft tissue in the mouth of a subject, or (c) both (a) and (b);
applying the dental material onto the gum or the soft tissue in the mouth; and
allowing the adhesive to cure and fix the dental material to the gum or the soft tissue in the mouth, whereupon the dental material is bonded (i.e. affixed) to the gum or the soft tissue in the mouth.

Further provided is a method for bonding a dental material to a gum or a soft tissue in a mouth, which method comprises:
applying an adhesive comprising a zein, a tannic acid, and a polymer either (a) to the dental material, (b) to the gum or the soft tissue in the mouth of a subject, or (c) both (a) and (b);
applying the dental material onto the gum or the soft tissue in the mouth; and
allowing the adhesive to cure and fix the dental material to the gum or the soft tissue in the mouth, whereupon the dental material is bonded to the gum or the soft tissue in the mouth.

The inorganic component and the polymer used in the adhesives are as defined above. The dental material can be a dental restorative or orthodontic appliance. In some embodiments, the dental material is a composite, a filling, or a sealant (e.g., a tooth sealant).

A method of scaling a damaged tissue of a mouth is also provided. The adhesive can act as a tissue sealant, for example. Provided is a method of sealing a wound or closing an incision in a gum or a soft tissue of a mouth of a subject, which method comprises: applying an adhesive comprising (i) a zein, (ii) a tannic acid, and (iii) an inorganic component to the wound or the incision in the gum or the soft tissue of the mouth (e.g., in an oral environment comprising at least saliva); and allowing the adhesive to cure, whereupon the wound is sealed or the incision is closed;

Still further provided is a method of sealing a wound or closing an incision in a gum or a soft tissue of a mouth, which method comprises: applying an adhesive comprising (i) a zein, (ii) a tannic acid, and (iii) a polymer to the wound or the incision in the gum or the soft tissue of the mouth (e.g., in an oral environment comprising at least saliva); and allowing the adhesive to cure, whereupon the wound is sealed or the incision is closed.

The adhesives can provide good retention and sealing. The adhesives can be prepared by mixing a zein stock solution, a tannic acid, and optionally a filler such as an inorganic component, or a polymer in an aqueous alcoholic solvent. The zein stock solution can be prepared by dissolving a zein powder in an aqueous solution of ethanol (ethanol+water) and adjusting the pH of the solution to about 4-11 using a pH modifier, such as a sodium hydroxide (NaOH) solution. A method of preparing wet-setting adhesives for an oral environment (e.g., of a subject) comprises:
a. preparing a zein stock solution by mixing the zein with a solution containing alcohol and water;
b. adjusting the pH of the zein solution to 4-11;
c. mixing the zein solution with a tannic acid in the presence of alcohol to obtain a highly viscous formulation; and
d. optionally mixing the highly viscous formulation with a filler in the presence of alcohol to obtain an opaque, a coacervate, a paste, or a putty-like mixture.

The adhesives can further comprise alcohol and water and be viscous, wherein the viscosity of the composition ranges from low to high or is in a solid form. The adhesive can be cured upon removal of the solvent. The solvent can be removed from the adhesive by exposing the adhesive to oxygen. The solvent can be removed from the adhesive by exposing the adhesive to heat. The alcohol can be ethanol, isopropyl alcohol, or any other alcohol that may be appropriate for the desired use-case of the adhesive. In some embodiments, the solvent is ethanol.

An aspect hereof is to provide an adhesive that provides strong bonding in an oral environment at body temperature, such as about 37° C. The adhesive can also stick well in the presence of a biofilm. Biofilm formation or surface fouling on substrate surfaces generally negatively influences the strength of adhesion and thus can significantly decrease bonding strength. These biofilms are a problem for dental adhesives that work in the presence of saliva.

The saline solution and the artificial saliva were prepared to mimic the oral environment. The saline solution (a mixture of salt water) containing 0.9 percent sodium chloride (salt) was prepared. The artificial saliva was prepared using a modified neutral phosphate buffer by saturating it with porcine mucin. The substrates play an important role in testing bonding in the oral environment. Teeth are comprised of two very different substrates: enamel and dentin. Tooth enamel is made of mostly hydroxyapatite and some percentage of calcium carbonate. Enamel is essentially 96% hydroxyapatite, whereas dentin consists of 70% hydroxyapatite. Hydroxyapatite was used to mimic enamel, stainless steel was used for dental repair or a dental appliance, and collagen was used to mimic the oral mucosa. Bronze surfaces are lighter, easier to handle in aqueous environments, and can provide some biocidal properties when working in artificial saliva at 37° C. and over several days. In some embodiments, the substrate is selected from limestone, hydroxyapatite (enamel mimic), stainless steel (dental repair), bronze, and collagen (skin or oral mucosa mimic). In some embodiments, the adhesion strength is substrate-specific as well as composition-specific.

The adhesive comprising an inorganic component can work well in artificial saliva on substrates such as hydroxyapatite and collagen substrates. The artificial saliva mimics the oral environment, and the hydroxyapatite and collagen substrates mimic the dental surface. In some embodiments, the adhesive comprising laponite can perform best on hydroxyapatite surfaces in saline solution and in artificial saliva, whereas the adhesive comprising CaCO₃ or albumin performed better on stainless steel.

In some embodiments, the adhesive comprising an inorganic component has adhesive strength/value at 37° C. in the saline solution of about 0.05 MPa to about 0.5 MPa, such as about 0.05 MPa to 0.5 MPa or 0.05 MPa to about 0.5 MPa or 0.05 MPa to 0.5 MPa. In some embodiments, the adhesive comprising an inorganic component, e.g., LRD has adhesive strength/value of about 0.1 MPa to about 0.5 MPa, such as about 0.1 MPa to 0.5 MPa or 0.1 MPa to about 0.5 MPa or 0.1 MPa to 0.5 MPa. In some embodiments, the adhesive comprising an inorganic component, e.g., XLS has adhesive strength/value of about 0.05 MPa to about 0.5 MPa, such as about 0.05 MPa to 0.5 MPa or 0.05 MPa to about 0.5 MPa or 0.05 MPa to 0.5 MPa. In some embodiments, the adhesive comprising an inorganic component, e.g., CaCO₃ has adhesive strength/value of about 0.06 MPa to about 0.5 MPa, such as about 0.06 MPa to 0.5 MPa or 0.06 MPa to about 0.5 MPa or 0.06 MPa to 0.5 MPa. In some embodiments, the adhesive comprising a polymer has adhesive strength/value of about 0.04 MPa to about 0.3 MPa, such as about 0.04 MPa to 0.3 MPa or 0.04 MPa to about 0.3 MPa or 0.04 MPa to 0.3 MPa. All ranges set forth in this paragraph are inclusive of the stated end points and all 0.05 MPa increments encompassed thereby.

In some embodiments, the adhesive comprising an inorganic component e.g., LRD has adhesive strength/value at 37° C. in the artificial saliva of about 0.05 MPa to about 0.3 MPa, such as about 0.05 MPa to 0.3 MPa or 0.05 MPa to about 0.3 MPa or 0.05 MPa to 0.3 MPa. In some embodiments, the adhesive comprising an inorganic component, e.g., CaCO₃ has adhesive strength/value at 37° C. in the artificial saliva of about 0.08 MPa to about 0.2 MPa, such as about 0.08 MPa to 0.2 MPa or 0.08 MPa to about 0.2 MPa or 0.08 MPa to 0.2 MPa. In some embodiments, the adhesive comprising an inorganic component, e.g., ZnO has adhesive strength/value at 37° C. in the artificial saliva of about 0.02 MPa to about 0.3 MPa, such as about 0.02 MPa to 0.3 MPa or 0.02 MPa to about 0.3 MPa or 0.02 MPa to 0.3 MPa. All wt ranges are inclusive of the stated end points and all 0.05 MPa increments encompassed thereby.

The adhesives can also work as primers for coating a cavity wall before the tooth cavity is filled to provide satisfactory adhesive strength to the tooth. In some embodiments, the adhesive coating comprises (i) a zein, (ii) a tannic acid, and (iii) optionally a filler.

Provided is a method of forming a coating on a tooth surface, which method comprises:
applying an adhesive comprising (i) a zein, (ii) a tannic acid, and (iii) optionally a filler to the tooth surface; and
allowing the adhesive to cure, whereupon the adhesive forms a coating on the tooth surface.

The adhesive can be applied to form a coating on the tooth surface after filling a tooth cavity, treating a tooth decay, or performing a root canal.

In some embodiments zein is at or about 25-45 wt % and the tannic acid present in at or about 1-25 wt % of the cured coating. In some embodiments, the amount of zein is at or about 25 wt %. In some embodiments, the amount of zein is at or about 26 wt %. In some embodiments, the amount of zein is at or about 30 wt %. In some embodiments, the amount of zein is at or about 35 wt %. In some embodiments, the amount of zein is at or about 40 wt %. In some embodiments, the amount of zein is at or about 45 wt %. In some embodiments, the amount of tannic acid is at or about 1 wt %. In some embodiments, the amount of tannic acid is at or about 7 wt %. In some embodiments, the amount of tannic acid is at or about 14 wt %. In some embodiments, the amount of tannic acid is at or about 21 wt %. In some embodiments, the amount of tannic acid is at or about 28 wt %. In some embodiments, the amount of tannic acid is at or about 35 wt %. All wt % set forth in this paragraph reference the wt % of the uncured adhesive and all ranges are inclusive of the stated end points and all 1% increments encompassed thereby.

In some embodiments, the filler is an inorganic component. The inorganic components used in the adhesives are as defined above. The inorganic component can be selected from natural clay, CaCO₃, ZnO, synthetic clay, and any combination thereof. In some embodiments, the inorganic component is selected from Montmorillonite (MMT-K10); Montmorillonite, dimethyl dialkyl amine (MMT-DDA or MMT-amine); Montmorillonite, trimethyl stearyl ammonium (MMT-TSA or MMT-am); Laponite® RD (LRD); Laponite® XLS (XLS); ZnO, and CaCO₃. Desirably, the inorganic component is ZnO. The incorporation of ZnO can improve the coating retention of the adhesive on the teeth surface.

In some embodiments, the filler is a polymer. The polymer can be selected from protein, polysaccharide, and a combination thereof. In some embodiments, the polymer is selected from casein, albumin, soy, gelatin, mucin, hydroxypropyl methyl cellulose, methyl cellulose, α-cellulose, Avicel pH-10, and any combination thereof.

The adhesive coatings hereof were tested on various substrates in the presence of artificial saliva. The substrate used in at least one study was hydroxyapatite-coated glass and the adhesive was cured in the presence of artificial saliva. The adhesive coatings tested were:
(i) Z4017, wherein the amount of zein present in the coating can be at or about 40 wt % and the amount of tannic acid present can be at or about 7 wt %.
(ii) Z26T21, wherein the amount of zein present in the coating can be at or about 26 wt % and the amount of tannic acid present can be at or about 21 wt %.
(iii) Z35T14, wherein the amount of zein present in the coating can be at or about 35 wt % and the amount of tannic acid present can be at or about 14 wt %.
(iv) Z35T21, wherein the amount of zein present in the coating can be at or about 35 wt %, and the amount of tannic acid present can be at or about 21 wt %.

Adhesives Z35T14 and Z35T21 showed the best adhesion with about 98%-99% (e.g., 98%-99%) retention of coating. Z35T14 and Z35T21 were further tested by adding about 1 wt % to about 5 wt % of ZnO. The ZnO improved coating retention percentage from about 98-99% to about 99-100% (FIGS. 15A-15C). The tested adhesives exhibited higher bonding strength/adhesion when the substrates were cured under artificial saliva instead of in air.

The pharmaceutical carrier devices have limited applications in the mouth due to the presence of saliva. The adhesive of the present disclosure can also be used for the delivery of pharmaceuticals and other compounds to wet tissue surfaces e.g., mucosal surfaces in the mouth.

### EXAMPLES

The following examples serve to illustrate the present disclosure. The examples are not intended to limit the scope of the claimed invention in any way.

### Example 1

The adhesives that showed promising results underwater (seawater) and at room temperature were evaluated for saline solution and artificial saliva. The results suggested that adhesives applied in seawater and saline solution exhibit similar bond strength. The adhesive comprising zein and tannic acid showed maximum adhesive strength was used as a control.

The underwater adhesives evaluated for dental and oral applications are summarized in Table 1. Optimal wet adhesion at body temperature, such as 37° C. and ease of handling during the application of glue to wet and saliva-coated surfaces were criteria for selecting the adhesive compositions. The sample compositions of the adhesives in ethanol and water prior to application are listed on the left. The dry solid content of the adhesives are listed on the right. Percentage (%), refers to the weight percentage of dry solid materials without solvents.

TABLE 1

| | |
|---|---|
| In 5 g Ethanol-Water + | Dry Solid Content or |
| 1-2 g Ethanol for Dilution | Ratio Estimated |

| Zein | Tannic Acid | Zein | Tannic Acid |
|---|---|---|---|
| (g) | (g) | (wt %) | (wt %) |
| 2.2 | — | 100 | — |
| 2.2 | 0.1 | 96 | 4 |
| 2.2 | 0.3 | 88 | 12 |
| 2.2 | 0.5 | 81 | 19 |
| 2.2 | 1.0 | 69 | 31 |
| 2.2 | 1.5 | 60 | 40 |
| 2.2 | 2.0 | 52 | 48 |
| 2.2 | 2.5 | 47 | 53 |
| 2.2 | 3.0 | 42 | 58 |
| 2.2 | 3.5 | 39 | 61 |
| 2.2 | 4.0 | 35 | 65 |
| 2.2 | 5.0 | 30 | 70 |

Table 2 shows selected compositions of adhesives explored for dental and oral applications. Estimated dry solid contents in grams and weight percentage (wt %) are listed for zein, tannic acid, and fillers. The fillers can be selected from the inorganic components and polymers as described above. All adhesives were formulated in water-ethanol solutions with some extra added ethanol for diluting high viscous formulations.

TABLE 2

| Adhesive in ca. | | | Estimated dry solid | | |
|---|---|---|---|---|---|
| 5-10 g ethanol-water | | | content or ratio | | |
| zein | tannic acid | Filler | zein | tannic acid | Filler |
| (g) | (g) | (g) | (wt %) | (wt %) | (wt %) |
| 2.2 | — | — | 100 | — | — |
| 2.2 | 3.0 | — | 42 | 58 | — |
| 2.2 | 3.0 | 0.25 | 40 | 55 | 5 |
| 2.2 | 3.0 | 1.0 | 36 | 48 | 16 |
| 2.2 | 3.0 | 2.0 | 30 | 42 | 28 |
| 2.2 | 3.0 | 2.5 | 29 | 39 | 32 |
| 2.2 | 3.0 | 3.0 | 27 | 36.5 | 36.5 |
| 2.2 | 3.0 | 3.5 | 26 | 34 | 40 |
| 2.2 | 3.0 | 4.0 | 24 | 33 | 43 |
| 2.2 | 3.0 | 5.0 | 22 | 29 | 49 |
| 2.2 | 3.0 | 6 | 19 | 27 | 54 |
| 2.2 | 3.0 | 8 | 16 | 23 | 61 |

The first composition comprising zein (42 wt %) and tannic acid (58 wt %) with no filler was the control, which yielded maximum underwater adhesion strength (e.g., Schmidt et al., Underwater bonding with a biobased adhesive from tannic acid and zein protein, ACS Applied Material & Interfaces 15(27): 32863-32874 (2023), which is hereby specifically incorporated by reference for its teachings regarding the same).

### Materials:

Zein corn protein with a molecular weight of 19-22 kDa (19-22 kg/mol) and tannic acid (1.7 kg/mol) was purchased from Sigma Aldrich, and ethanol was obtained from Decon Laboratories. Laponite-type clays from Southern Clay or Talas, calcium carbonate (CaCO₃), or zinc oxide (ZnO). ZnO was obtained from Sigma Aldrich.

### Zein Stock Solution

Clear and amber-colored zein stock solutions were prepared by vigorously mixing zein powder (55.5 g) with a solution containing ethanol (45 g, 95% or 99%) and water (23 g, distilled or tap water). A premixed solution of ethanol and water was prepared to get an amber and a clear zein solution because zein does not dissolve in water; adding water first and then ethanol would lead to a heterogeneous mixture or slurry with many hardened zein clumps that will not easily dissolve. Sodium hydroxide (4 g, 10 M NaOH) solution was added to the zein solution under mixing to adjust the pH to 4-11. The resulting highly viscous zein solutions were all clear (see-through) and with different shades of amber.

### Zein-Tannic Acid Adhesive

1. Zein-tannic acid adhesive formulation was prepared by mixing the prepared zein stock solution (5 g of a solution containing about 2.2 g of dry zein powder) with tannic acid powder (1 g). Extra ethanol can be added while mixing as necessary to obtain a highly viscous putty or thick gel-like substance or a thick solution/dispersion. The adhesive is ready for application once it pulls the fibers.
2. Zein-tannic acid adhesive formulation was prepared by mixing the prepared zein stock solution (5 g of a solution containing about 2.2 g of dry zein powder) with tannic acid powder (3 g) (see Table 1). Extra ethanol (4 g) can be added while stirring to control viscosity and allow easier mixing when the inorganic component was added. The resulting amber transparent zein-tannic acid solution was used 24 hours after preparation.

Both zein stock solution and zein-tannic acid adhesive provided baseline controls. Adhesive dispersions were made from the zein-tannic acid solution described above, and an inorganic component was added. The inorganic component was selected from laponite, CaCO₃, and ZnO.

Adhesive compositions from zein and tannic acid were prepared according to Table 1. Adhesive compositions containing additional fillers, e.g., inorganic components or polymers, are summarized in Table 2. Most adhesive solutions, dispersions, and putties were used within 1-24 hours after preparation. The dispersions were hand-mixed again right before use to guarantee reproducibility. After more than 24 hours of drying on a benchtop, many dispersions hardened irreversibly. Depending on the composition and the amount of added ethanol, some of the ZnO, CaCO₃, and laponite-containing adhesive formulations hardened irreversible within 1 minute-1 hour. Once hardened, these materials could not be used for bonding.

### Zein-Tannic Acid-Inorganic Component Adhesive

(i) A zein stock solution (5 g) was first mixed with tannic acid powder (1 g). Ethanol was added to the mixture until it formed a highly viscous and translucent amber-colored solution. The pH was adjusted to about 9. (ii) inorganic component (0.1 g-7.0 g) powder was added to the highly viscous zein-tannic acid solution together with more ethanol to wet and manually mix the powder into the solution. The resulting mixture is opaque, coacervate-like or paste-like, or putty-like.

### Zein-Tannic Acid-CaCO₃ Adhesive

(i) A zein stock solution (5 g) was first mixed with tannic acid powder (1 g). Ethanol was added to the mixture until it formed a highly viscous and translucent amber-colored solution. The pH was adjusted to about 9. (ii) CaCO₃ (4.0 g) powder was added to the highly viscous zein-tannic acid solution together with more ethanol to wet and manually mix the powder into the solution. The resulting mixture is opaque, coacervate-like or paste-like, or putty-like.

### 2. Zein-Tannic Acid-Laponite Adhesive

(i) A zein stock solution (5 g) was first mixed with tannic acid powder (1 g). Ethanol was added to the mixture until it formed a highly viscous and translucent amber-colored solution. The pH was adjusted to about 8-9. (ii) laponite clay, (7.0 g) powder, was added to the highly viscous zein-tannic acid solution together with more ethanol to wet and manually mix the powder into the solution. The resulting mixture was opaque, coacervate-like or paste-like, or putty-like.

### Zein-Tannic Acid-Polymer Adhesive

A zein stock solution (5 g solution) was first mixed with tannic acid powder (3 g). Ethanol was added to the mixture until it formed a highly viscous and translucent amber-colored solution. The pH was adjusted to about 8-9. (ii) Polymer powder (0.1 g-5.0 g) was added to the highly viscous zein-tannic acid solution together with more ethanol to wet and manually mix the polymer powder into the solution. The resulting mixture is opaque, coacervate-like or paste-like, or putty-like. The mixture can be diluted with more ethanol if desired. The polymer can be a cellulose derivative or a soy derivative, or another polymer such as casein, mucin, gelatin, or albumin.

### Zein-Tannic Acid-Hydroxypropyl Methyl Cellulose Adhesive

A zein stock solution (5 g solution) was first mixed with tannic acid powder (3 g). Ethanol was added to the mixture until it formed a highly viscous and translucent amber-colored solution. The pH was adjusted to about 8-9. (ii) hydroxypropyl methyl cellulose powder (4.0 g) was added to the highly viscous zein-tannic acid solution together with more ethanol to wet and manually mix the polymer powder into the solution. The resulting mixture is opaque, coacervate-like or paste-like, or putty-like. The mixture can be diluted with more ethanol if desired.

### Lap-Shear-Testing:

An Instron 5544 Materials Testing System with a 2000 N load cell was used for lap shear experiments and for quantifying bond strength. The substrate or adherend dimensions were 1.2 cm×10 cm, and the overlap areas were 1.2×1.2 cm most of the time. Substrates were pulled apart at 2 mm per minute. At least 5 adherend pairs for every adhesive composition and control were lap shear tested, and averages with standard deviations were reported. The adhesive was applied to the substrates in the saline solution or in artificial saliva baths at 37° C. and immediately used for lap-shear testing.

### Artificial Saliva Solution

Artificial saliva was made by saturating modified neutral phosphate buffer with porcine mucin.

### Adhesion in Saline Solution

The saline solution was made from 9 grams of NaCl per liter in deionized water. All parts of the system were submerged in solution before use. Substrates were glued together and then cured in saline solution at 37° C. for 24 hours. After curing, lap shear testing was used to determine adhesion strength. The adhesive containing Z 42% and TA 58% was used as a control. Limestone, stainless steel, collagen, and hydroxyapatite were used as substrates. After lap shear testing, substrates made of limestone, stainless steel, and hydroxyapatite showed more cohesive than adhesive failure. Collagen substrates showed adhesive failure most of the time.

### Example 2

### Bonding in Saline

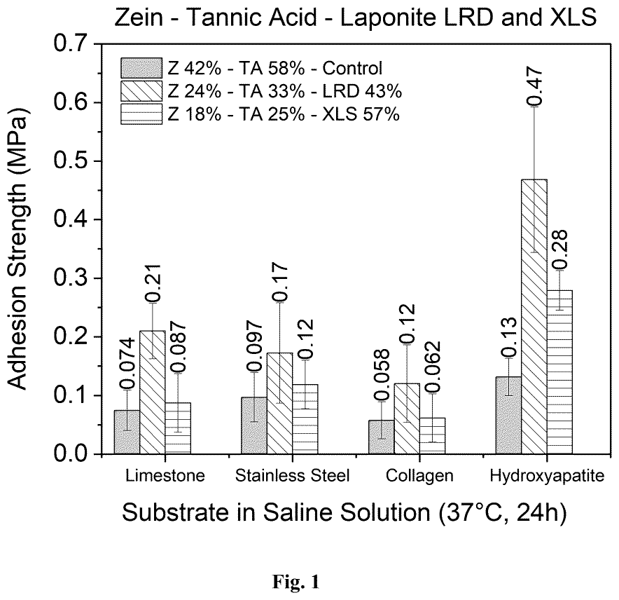

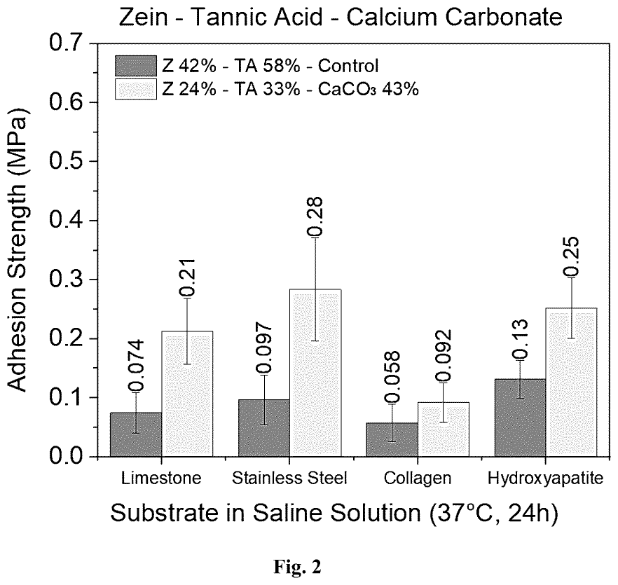

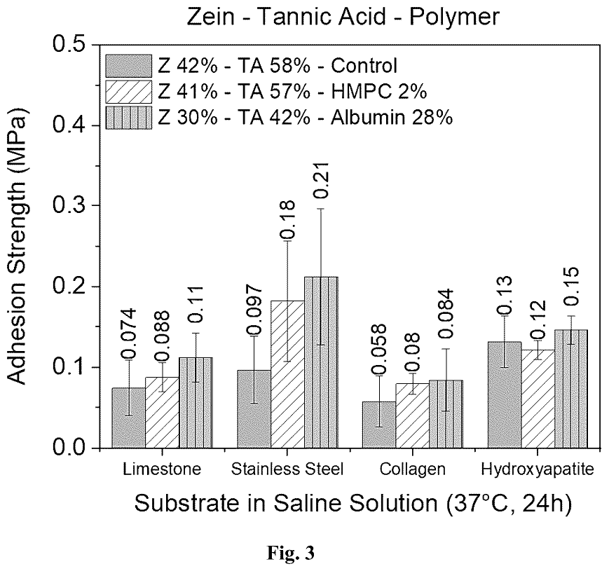

Adhesives tested for bonding in saline solution were:
(i) Z 24%-TA 33%-LRD 43%. LRD consists of pure clay silicate nanoplate.
(ii) Z 18%-TA 25%-XLS 57% Laponite® XLS is a polyphosphate-treated pure clay nanoplate. The adhesives comprising laponite performed best on the HAP surfaces with the pure LRD showing higher bond strength than the XLS polyphosphate-treated version at 37° C. These adhesives had stronger bonding than the control on all substrates (see FIG. 1). After lap shear testing, substrates made of limestone, steel and HAP showed more cohesive than adhesive failure. Collagen substrates showed adhesive failure most times.
(iii) Z 24%-TA 33%-CaCO₃ 43%. It was observed that the amount of calcium carbonate in this adhesive led to a maximum bond strength when compared to many other Z-TA-CaCO₃ compositions. The adhesives containing calcium carbonate showed different trends when compared to the adhesives containing laponite. Bond strengths were highest for stainless steel substrates and lower for HAP substrates. The addition of calcium carbonate made the adhesive better performing when compared to the Z-TA control (see FIG. 2). After lap-shear testing, limestone, steel and HAP substrates showed both adhesive and cohesive failure modes with a little more cohesive failure. Collagen substrates showed adhesive failure most times.
(iv) Z 41%-TA 57%-HPMC 2%, and (v) Z 30%-TA 42%-Albumin 28%. The results indicated that the adhesives containing a polymer perform better on stainless steel when compared to the other substrates. Bond strength was even better than that of adhesives containing laponite tested on stainless steel. Adhesion values on HAP surfaces were similar to the Z-TA control and lower when compared to the adhesives containing laponite and CaCO₃ (see FIG. 3).

Thus, the adhesion strength of the adhesives (i)-(v) was composition and substrate-dependent under saline solution and at constant temperature. For the adhesives containing laponite, bond strengths observed on limestone substrates were much lower than those on HAP coatings. This suggests that limestone is not a good HAP mimic for some adhesives.

### Example 3

### Bonding Under Artificial Saliva Solutions

The adhesion studies were carried out with substrates held under artificial saliva, and the glues were also held under these solutions when applied to surfaces. The artificial saliva solution was prepared as described above.

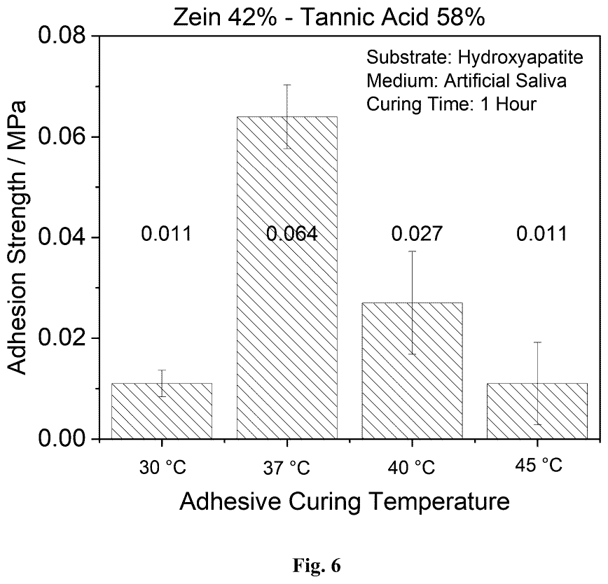

All experiments carried out in artificial saliva were done at about 37° C. to simulate body temperature. For some oral and dental applications, the temperature inside the oral cavity may fluctuate. Higher temperatures still tolerated in the mouth have been found to be around 45° C.-50° C. Because these temperature fluctuations are important for handling and applying adhesives, adhesion performance was evaluated for control adhesive of a Z-TA (Z 42%-TA 58%), cured for 1 hour at specific temperatures within a range of about 30° C.-45° C. Hydroxyapatite substrates were used in artificial saliva. FIG. 6 summarizes the initial findings. The results suggested that the composition had maximum adhesion at 37° C.+/−1° C.

### Adhesion for Zein-Tannic Acid in Artificial Saliva

The adhesion strengths on stainless steel and bronze can often be similar. Bronze substrates are lighter than steel, thus making them easier to handle with low-strength adhesives in aqueous environments such as artificial saliva. Bronze surfaces also provide some biocidal properties when working with biofilm-coated surfaces that were placed in saliva at 37° C. The polished bronze substrates were placed in artificial saliva at 37° C. for about 5 minutes before glue was applied. After the application of glue, substrates were pressed together and left in the artificial saliva bath for 1 hour before lap shear testing. A broad maximum in adhesion strength was observed for zein-tannic acid formulations listed in Table 1 that have 20-40 weight % tannic acid (FIG. 6).

### Adhesion for Zein-Tannic Acid-Inorganic Component in Artificial Saliva

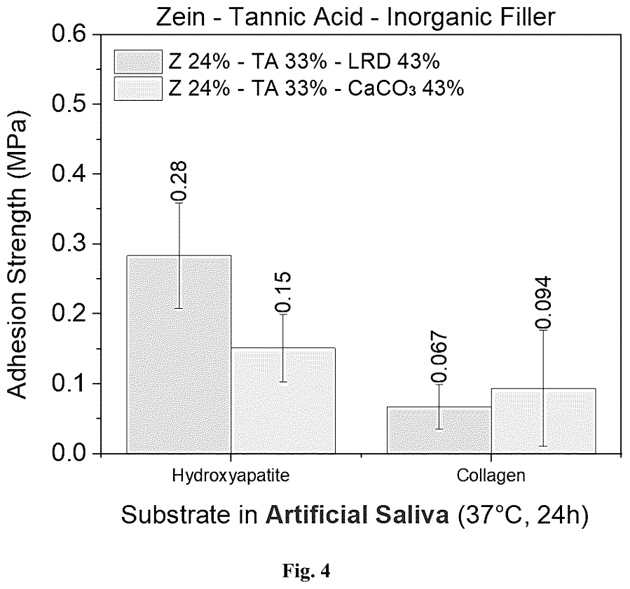

The adhesives containing inorganic components such as laponite and calcium carbonate were tested on HAP and collagen surfaces in the presence of artificial saliva at 37° C. The substrates were left in artificial saliva for 3 hours prior to applying adhesive. This was done to better mimic the oral environment. Samples were then applied to the substrates in saliva and left there for 24 hours to cure (see FIG. 4). FIG. 4 shows that adhesives containing laponite e.g., Z 24%-TA 33%-LRD 43% (Table 2) perform better on HAP surfaces than calcium carbonate containing adhesives Z 24%-TA 33%-CaCO₃ 43% with the similar composition by weight of solids. On collagen surfaces, both adhesives perform equally well within error bars.

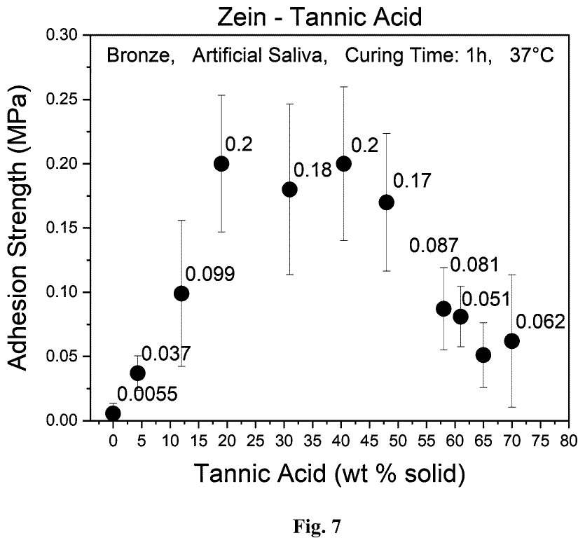

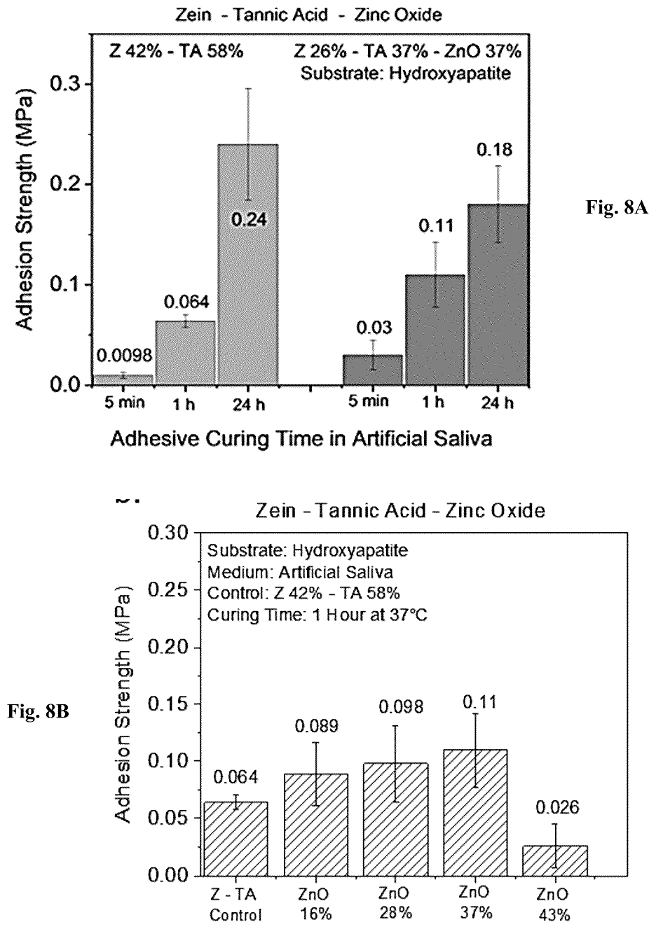

FIG. 8A compares the performance of adhesives Z-TA and Z-TA-ZnO. All adhesives were cured in artificial saliva at 37° C. and on HAP substrates. Substrates were soaked in artificial saliva for an hour before adhesive was added. After the application of glue, substrates were pressed together and left in the artificial saliva bath for either 5 minutes, 1 hour, or 24 hours before lap shear testing. Glued substrates were pulled apart via lap shear adhesion testing. The sample compositions used were Z 42%-TA 58% and Z 26%-TA 37%-ZnO 37%. The general trends in adhesion indicate that the longer the adhesives are cured, the higher the bond strengths. The Z-TA adhesive appears to perform somewhat better when compared to the Z-TA-ZnO adhesive after 24 hours of curing time. At 5 minutes and 1 hour curing time, the ZnO-containing adhesive performs slightly better. ZnO increases bond strengths from about 0.064 MPa no ZnO to about 0.11 MPa with ZnO for short curing times. The main difference between these two adhesives (FIG. 7A) is the hardening during sample preparation. While a Z-TA solution can be kept for several months in a capped vial on benchtop, once ZnO has been added, the adhesive hardens irreversibly, even when kept in a capped container. The Z-TA-ZnO samples were best prepared about 5 minutes before application to the substrates.

FIG. 8B depicts adhesion strength as a function of zinc oxide content for Z-TA adhesives with Z 42%-TA 58% composition as control. Table 2 lists the compositions for each sample tested. Overall, it was observed that ZnO initially increases bond strength, and the bell curve is consistent with trends observed for other inorganic components. The best-performing adhesive was at around Z 26%-TA 37%-ZnO 37%. Higher ZnO concentrations, such as 44%, lead to a decrease in bond strength. The rapid curing and hardening of the ZnO-containing adhesive compared to the adhesive containing other inorganic components may be related to the deprotonation of ZnO, which, in a basic environment, aids the oxidation of gallol groups present in the tannic acid. These oxidation processes lead to cross-linking and darkening of the adhesive, which was observed visually. The adhesive that is exposed to oxygen from air darkens more and faster, while the adhesive that hardens while in contact with the plastic container surface maintains a light color.

### Adhesion for Zein-Tannic Acid-Laponite on Hydroxyapatite in Artificial Saliva

Knowing how bond strengths changes as a function of curing time is important to dental materials development. Bonding at short cure times is desired, but rapid adhesion and quick hardening does not mean that the highest bond strength is achieved at the moment of application. Initial adhesion to hold the substrates in place can be followed by gradual curing.

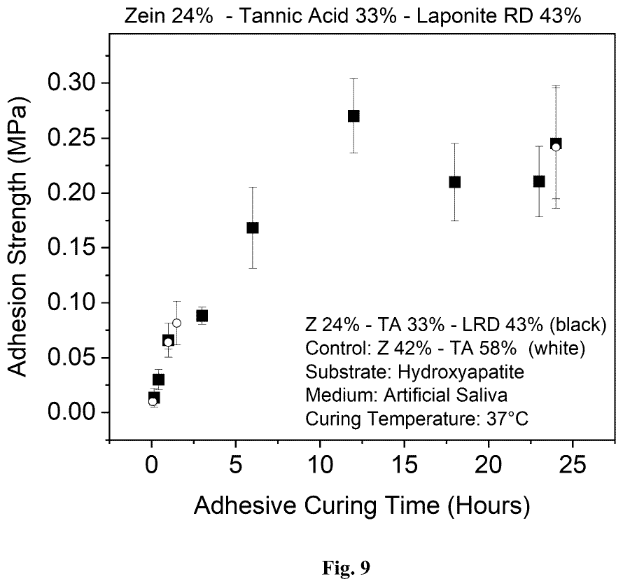

An adhesive Z-TA-LRD (Z 24%-TA 33%-LRD 43%) was prepared according to Table 2. The Z-TA-LRD adhesive dispersions were used within 1-24 hours after preparation. The amber dispersions were hand-mixed right before use to guarantee reproducibility. After more than 24 hours, the dispersions hardened irreversibly when left on a benchtop. FIG. 9 shows the adhesion strength as a function of adhesive curing time in artificial saliva. Curing times tested were between 5 minutes and 24 hours. The glued hydroxyapatite substrates were cured for a specific time in artificial saliva at 37° C.

### Example 4

### Biofilm Formation and Adhesion for Zein-Tannic Acid in Artificial Saliva

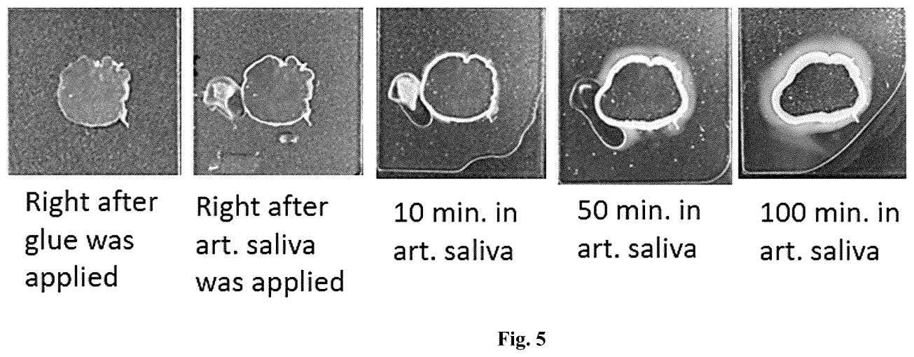

Biofilm formation or surface fouling on substrate surfaces generally influences adhesion and significantly decreases bonding strength. These biofilms are a problem for dental adhesives that must work in the presence of saliva. Although adhesives that stick to biofilms, at warm temperatures are rare or nonexistent, it was found that there is some adhesion possible. Bond strengths were not as high as those measured in saline solutions. The data were reproducible when exact handling procedures were followed. Saliva-coated collagen proved to be a difficult substrate to work with. Experiments in artificial saliva showed that keeping the adhesive in warm artificial saliva for about 10 seconds before applying this to the collagen substrates allowed for better adhesion and better manipulation of the material between substrate surfaces. To better understand and optimize adhesive application and manipulation in artificial saliva, the curing behavior of adhesive drops sandwiched between glass slides and exposed to artificial saliva (FIG. 5) was visually observed. The experiments were done at room temperature, where the evaporation of artificial saliva could be controlled by adding deionized water. Adhesive curing was slow enough to be seen, and photos could be taken to document curing with time. Once the amber-colored zein-tannic acid adhesive was placed underwater or in solution, a skin or outer layer formed on the exterior within seconds. Initially this coating was grey yellow and opaque and harder than the inside liquid adhesive. The zein protein within the glue is not soluble in water and immediately phase separates when in contact with aqueous solutions, thereby explaining the formation of the skin. Inside the exterior layer resides the amber adhesive temporarily protected from the aqueous surrounding. When an adhesive drop is sandwiched between microscopy glass slides, the formation and growth of the exterior layer around the drop can be visualized as a ring becoming thicker (FIG. 5). Growth of the exterior layer and the resulting color changes can be correlated with adhesive hardening and curing times.

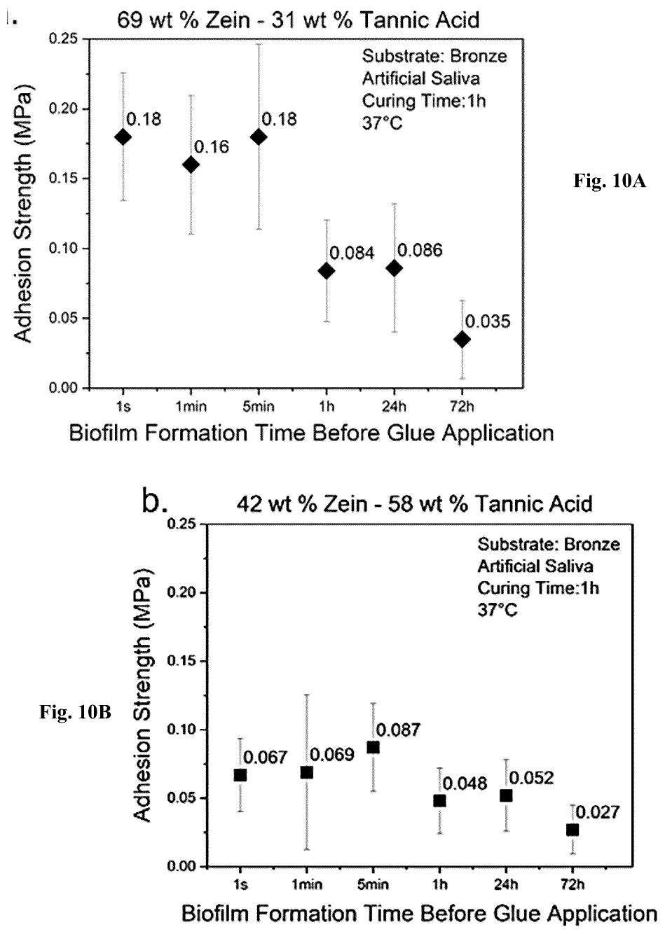

The strongest adhesive compositions at 0.18 MPa was observed for 69 wt % zein-31 wt % tannic acid. For comparison also evaluated a lower performing composition at about 0.07 MPa for 42 wt % zein-58 wt % tannic acid. This lower-performing composition has nearly double the amount of tannic acid when compared to the high-performing one. The idea was to determine if a higher tannic acid concentration would allow for maintaining bond strength in the presence of biofilms. FIGS. 10A and 10B suggest that increased biofilm formation decreases bonding strength after about 5 minutes of biofilm formation on the substrates for the high-performing adhesive at 69 wt % zein-31 wt % tannic acid (FIG. 10A). After 72 hours of biofilm formation adhesion is still possible but decreased fivefold. The lower performing adhesive maintained bond strength better but within error bars (FIG. 10B). The trends suggest that the tannic acid is influencing adhesion strength in the presence of biofilms.

### Example 5

### Effect of Coating Thickness on Coating Adhesion Strength

The adhesion of adhesive coatings was also studied on HAP-coated glass substrates. The coatings were cured under saliva for 10 minutes, and testing was performed at pressure setting 5. When the adhesive was applied as a thicker layer, the coating retention was poor, but when the adhesive was applied as a thinner layer, the coatings retained much better.

### Example 6

### Effect of Zein to Tannic Acid Ratio on Coating Adhesion Strength

Different formulations were prepared and tested to determine the best zein-to-tannic acid ratio for maximum coating adhesion strength. The substrates HAP coated glass substrates were immersed in saliva for 1 hour at 37° C. The formulations were then coated on a 1.2 cm×1.2 cm area on the substrate using a spatula. Four different formulations were cured on each substrate (FIG. 13). They were cured for 5 min in air and then tested using a water flosser at a pressure setting of 1 for 2 minutes. Table 3 shows the percentage of each adhesive formulation retained on substrates. Z35T14 and Z35T21 appeared to be the best adhesive formulation overall, with over 98% retention.

Table 3 shows the percentage of adhesive coatings retained on substrates after the water flosser test at a pressure setting 1.

TABLE 3

| Adhesive (wt %) | % retention | Adhesive (wt %) | % retention |
|---|---|---|---|
| 26% Z-7% TA | 50-90 | 35% Z-1% TA | 80-90 |
| 26% Z-14% TA | 80-95 | 35% Z-7% TA | 90-95 |
| 26% Z-21% TA | 15-20 | 35% Z-14% TA | 98-99 |
| 26% Z-28% TA | 20-15 | 35% Z-21% TA | 98-99 |
| 30% Z-1% TA | 90-95 | 40% Z-1% TA | 90-98 |
| 30% Z-7% TA | 50-90 | 40% Z-7% TA | 60-95 |
| 30% Z-14% TA | 50-90 | 40% Z-14% TA | 60-70 |
| 30% Z-21% TA | 10-40 | 45% Z-1% TA | 50-80 |
| | | 45% Z-7% TA | 40-60 |

### Example 7

Adhesion of Commercial Dental Coatings Vs. Adhesive Coating

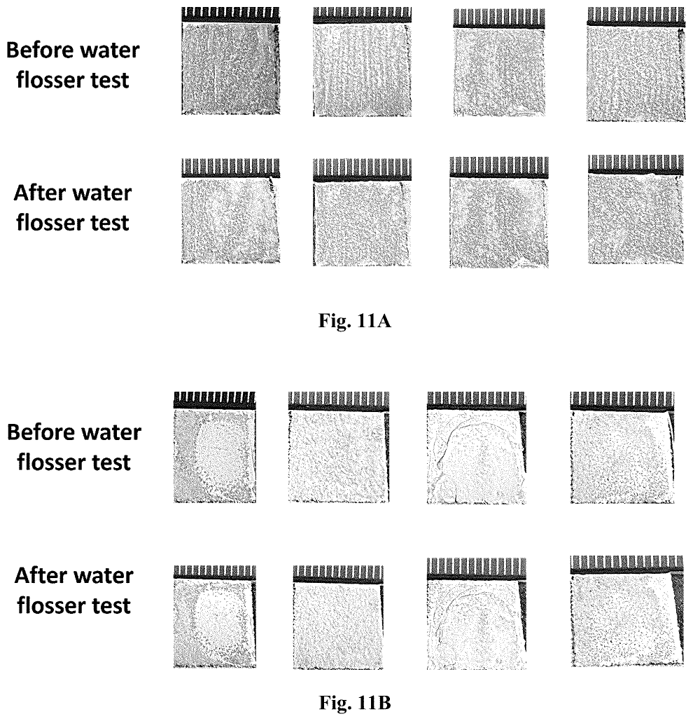

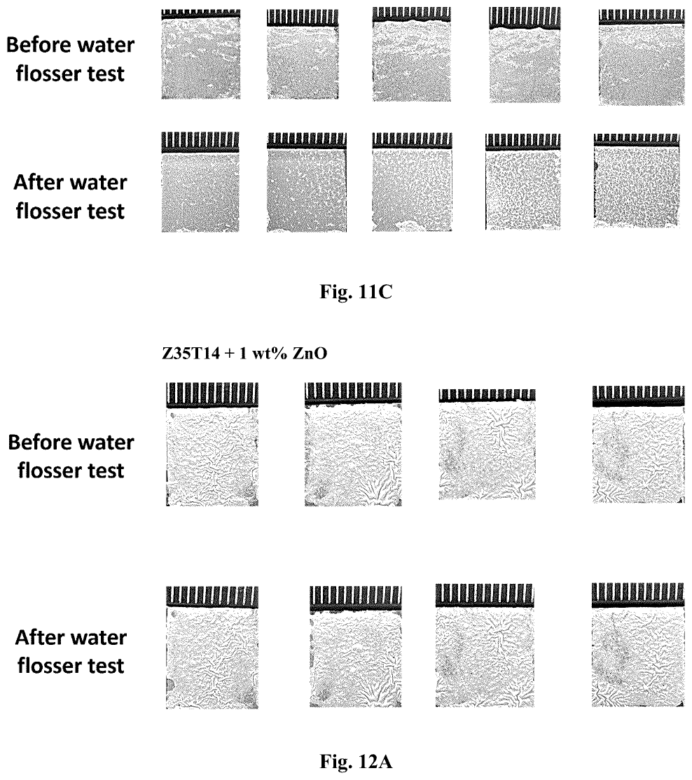

Three commercial dental coatings/adhesives were studied to compare their adhesion strength to the adhesive coatings. PreviDent® varnish (FIG. 11A) and Colgate® Optic White® Overnight Teeth Whitening Pen (FIG. 11B) showed excellent adhesion and had 100% coating retention after water flosser tests. Poligrip® denture adhesive showed poor adhesion when tested using a water flosser and had almost no coating retention (FIG. 11C). The adhesive was coated on a substrate of size of about 1.2 cm×1.2 cm.

### Example 8

### Incorporation of ZnO in Adhesives Z35T14 and Z35T21

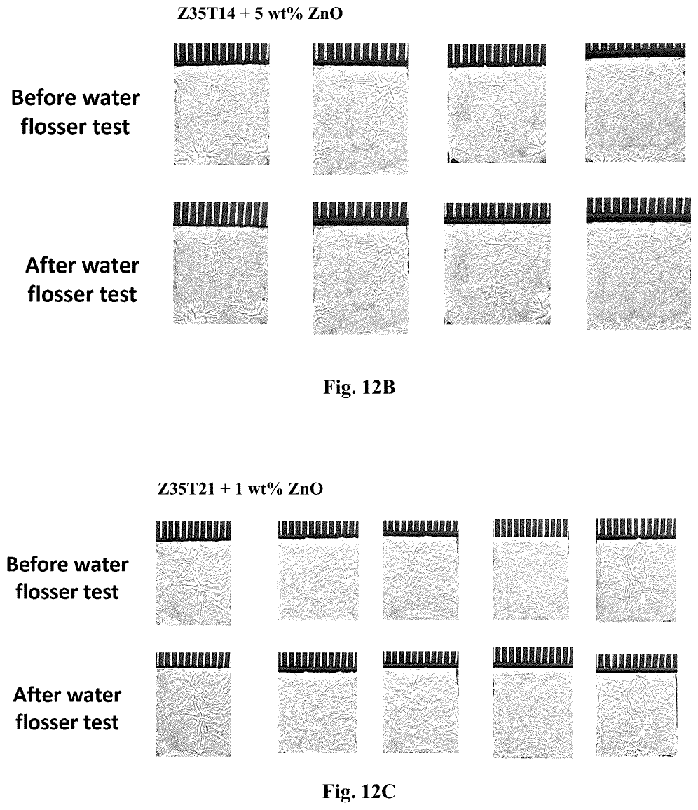

ZnO (1 and 5 wt %) was added as a filler to Z35T14 and Z35T21 adhesive formulations. The incorporation of ZnO improved the coating retention percentage of both Z35T14 and Z35T21 formulations from 98-99% to 99-100% (FIGS. 12A-C). The experiment could not be performed with Z35T21+5 wt % ZnO since the formulation was a putty consistency and could not be coated evenly.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% 99.5%, 99.9%, 99.99%, or 99.999% or more of a stated value or of a stated limit of a range, inclusive of the specified end points.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms.

When ranges are used herein for physical properties, such as weight percentages, or chemical properties, such as chemical formulae, all combinations and sub-combinations of ranges and specific embodiments therein are intended to be included.

Those skilled in the art will recognize that numerous modifications can be made to the specific implementations described above. The implementations should not be limited to the particular limitations described. Other implementations may be possible.

It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

All patents, patent application publications, journal articles, textbooks, and other publications mentioned in the specification are indicative of the level of skill of those in the art to which the disclosure pertains.

We claim:

1. A method for bonding a dental material to a tooth surface, which method comprises:
applying an adhesive to either (a) the tooth surface, (b) the dental material, or (c) both (a) and (b), the adhesive comprising a zein, a tannic acid and, optionally, a filler;
applying the dental material onto the tooth surface; and
allowing the adhesive to cure and fix the dental material to the tooth surface, whereupon the dental material is bonded to the tooth surface.

2. The method of claim 1, wherein the dental material is a dental restorative or an orthodontic appliance.

3. The method of claim 2, wherein the dental restorative is a composite, a filling, a sealant, an inlay, an onlay, a crown, or a bridge.

4. The method of claim 3, wherein the orthodontic appliance is a bracket, a crown, a buccal tube, a band, a cleat, a button, a lingual retainer, or a lingual bar.

5. The method of claim 1, wherein the filler is an inorganic component.

6. The method of claim 1, wherein the filler is a polymer.

7. The method of claim 5, wherein the inorganic component is selected from a group consisting of natural clay, calcium carbonate (CaCO₃), zinc Oxide (ZnO), synthetic clay, and any combination of the foregoing.

8. The method of claim 7, wherein the inorganic component is selected from a group consisting of Montmorillonite (MMT-K10); Montmorillonite, dimethyl dialkyl amine (MMT-DDA or MMT-amine); Montmorillonite, trimethyl stearyl ammonium (MMT-TSA or MMT-am); Laponite® RD (LRD); Laponite® XLS (XLS); ZnO; and CaCO₃.

9. The method of claim 6, wherein the polymer is selected from protein, polysaccharide, and a combination thereof.

10. The method of claim 9, wherein the polymer is selected from a group consisting of casein, albumin, soy, gelatin, mucin, hydroxypropyl methyl cellulose, methyl cellulose, α-cellulose, Avicel pH-10, and any combination of two or more of the foregoing.

11. A method for bonding a dental material to a gum or a soft tissue in a mouth, which method comprises:
applying an adhesive comprising a zein, a tannic acid and, optionally, a filler to: (a) the dental material, (b) the gum or the soft tissue in the mouth, or (c) both (a) and (b);
applying the dental material onto the gum or the soft tissue in the mouth; and
allowing the adhesive to cure and fix the dental material to the gum or the soft tissue in the mouth, whereupon the dental material is bonded to the gum or the soft tissue in the mouth.

12. The method of claim 11, wherein the filler is an inorganic component.

13. The method of claim 11, wherein the filler is a polymer.

14. The method of claim 12, wherein the inorganic component is a natural clay, CaCO₃, ZnO, a synthetic clay, or any combination of two or more of the foregoing.

15. The method of claim 14, wherein the inorganic component is selected from a group consisting of Montmorillonite (MMT-K10); Montmorillonite, dimethyl dialkyl amine (MMT-DDA or MMT-amine); Montmorillonite, trimethyl stearyl ammonium (MMT-TSA or MMT-am); LRD; XLS; ZnO; and CaCO₃.

16. The method of claim 13, wherein the polymer is selected from a group consisting of a protein, a polysaccharide, and a combination of a protein and a polysaccharide.

17. The method of claim 16, wherein the polymer is selected from the group consisting of casein, albumin, soy, gelatin, mucin, (hydroxypropyl)methyl cellulose, methyl cellulose, α-cellulose, Avicel pH-10, and any combination of two or more of the foregoing.

18. The method of claim 11, wherein the dental material is a composite, a filling, or a sealant.

19. A method of sealing a wound or closing an incision in a gum or a soft tissue of a mouth, which method comprises:
applying an adhesive comprising a zein, a tannic acid, and an inorganic component to the wound or the incision in the gum or the soft tissue of the mouth; and
allowing the adhesive to cure, whereupon the wound is sealed or the incision is closed.

20. A method of sealing a wound or closing an incision in a gum or a soft tissue of a mouth, which method comprises:
applying an adhesive comprising a zein, a tannic acid, and a polymer to the wound or the incision in the gum or the soft tissue of the mouth; and
allowing the adhesive to cure, whereupon the wound is sealed or the incision is closed.

21. A method of forming a coating on a tooth surface, which method comprises:
applying an adhesive comprising a zein, a tannic acid, and, optionally, a filler to the tooth surface; and
allowing the adhesive to cure, whereupon the adhesive forms a coating on the tooth surface.

22. The method of claim 21, wherein zein is at or about 25-45 weight percent and the tannic acid present in at or about 1-25 weight percent of the cured coating.

23. The method of claim 21, wherein the adhesive is applied after filling a tooth cavity, treating a tooth decay, or performing a root canal on the tooth surface.

24. The method of claim 21, wherein the filler is an inorganic component selected from a group consisting of natural clay, CaCO₃, ZnO, a synthetic clay, or any combination of two or more of the foregoing.

25. The method of claim 24, wherein the inorganic component is selected from the group consisting of Montmorillonite (MMT-K10); Montmorillonite, dimethyl dialkyl amine (MMT-DDA or MMT-amine); Montmorillonite, trimethyl stearyl ammonium (MMT-TSA or MMT-am); LRD, XLS; ZnO; and CaCO₃.

26. The method of claim 21, wherein the filler is a polymer selected from the group consisting of a protein, a polysaccharide, and a combination of a polymer and a polysaccharide.

27. The method of claim 26, wherein the polymer is selected from the group consisting of casein, albumin, soy, gelatin, mucin, (hydroxypropyl)methyl cellulose, methyl cellulose, α-cellulose, and Avicel pH-10, and any combination of two or more of the foregoing.
